(19) United States
(12) Patent Application Publication
(10) Pub. No.: US 2012/0136222 A1
(43) Pub. Date: May 31, 2012

(54) Methods And Systems For Monitoring A Ventilator Patient With A Capnograph

(75) Inventors: Peter Doyle, Vista, CA (US); Joseph Doug Vandine, Manteca, CA (US); Warren Sanborn, Escondido, CA (US); Dan Graboi, Encinitas, CA (US)

(73) Assignee: Nellcor Puritan Bennett LLC, Boulder, CO (US)

(21) Appl. No.: 12/956,238

(22) Filed: Nov. 30, 2010

(51) Int. Cl. A61B 5/0205; A61M 16/00

(52) U.S. Cl. 600/301; 128/204.23

(57) ABSTRACT

This disclosure describes systems and methods for monitoring the ventilation of a patient being ventilated by a medical ventilator. The disclosure describes a novel approach of displaying integrated ventilator information with capnography data. The disclosure further describes a novel approach for removing cardiogenic artifacts.

### BACKGROUND

Medical ventilator systems have been long used to provide supplemental oxygen support to patients. These ventilators typically comprise a source of pressurized oxygen which is fluidly connected to the patient through a conduit. Some ventilator systems monitor the patient during ventilation. In some systems, carbon dioxide (CO₂) levels in the breathing gas from the patient are measured.

Many of these previously known medical ventilators display the monitored CO₂ levels of the breathing gas from the patient. While these previously known ventilation systems display CO₂ readings or capnography data, patient care could be improved by further coordinating the operation of the two devices, particularly by integrating the analysis, storage and display of particular aspects of carbon dioxide data and other cardio-pulmonary data.

### SUMMARY

This disclosure describes systems and methods for monitoring the ventilation of a patient being ventilated by a medical ventilator. The disclosure describes a novel approach of displaying integrated ventilator information with capnography data. The disclosure further describes a novel approach for removing cardiogenic artifacts.

In part, this disclosure describes a method for monitoring the ventilation of a patient being ventilated by a medical ventilator-capnograph system. The method includes:

a) monitoring a pulse rate of a patient being ventilated by a medical ventilator-capnograph system with at least one of a flow sensor, a pressure sensor, a cardiac monitor, and an oximeter;

b) monitoring the patient with a capnograph, the capnograph monitors an amount of carbon dioxide in respiration gas from the patient to derive a capnogram;

c) determining potential cardiogenic artifacts of the capnogram;

d) correlating the potential cardiogenic artifacts of the capnogram with the pulse rate of the patient to verify cardiogenic artifacts of the capnogram; and

e) removing verified cardiogenic artifacts of the capnogram.

Yet another aspect of this disclosure describes a medical ventilator-capnograph including:

a) a pneumatic gas delivery system, the pneumatic gas delivery system adapted to control a flow of gas from a gas supply to a patient via a ventilator breathing circuit;

b) at least one sensor, the at least one sensor monitors a pulse rate of the patient;

c) a capnograph, the capnograph monitors an amount of carbon dioxide in respiration gas from the patient in the ventilator breathing circuit to generate a capnogram;

d) a correlation module, the correlation module is adapted to identify potential cardiogenic artifacts of the capnogram, correlate the potential cardiogenic artifacts with the pulse rate of the patient, and remove verified cardiogenic artifacts of the capnogram; and

e) a processor in communication with the pneumatic gas delivery system, at least one sensor, the capnograph, and the correlation module.

The disclosure further describes a computer-readable medium having computer-executable instructions for performing a method for monitoring the ventilation of a patient being ventilated by a medical ventilator-capnograph system. The method includes:

a) repeatedly monitoring a pulse rate of a patient being ventilated by a medical ventilator-capnograph system;

b) repeatedly monitoring carbon dioxide in breathing gas from the patient to derive a capnogram;

c) repeatedly determining potential cardiogenic artifacts of a capnogram;

d) repeatedly correlating the potential cardiogenic artifacts of the capnogram with the pulse rate of the patient to verify cardiogenic artifacts of the capnogram; and

e) repeatedly removing verified cardiogenic artifacts of the capnogram.

The disclosure also describes a medical ventilator-capnograph system, including means for monitoring a pulse rate of a patient being ventilated by a medical ventilator-capnograph system, means for monitoring carbon dioxide in breathing gas from the patient to derive a capnogram, means for determining potential cardiogenic artifacts of a capnogram, means for correlating the potential cardiogenic artifacts of the capnogram with the pulse rate of the patient to verify cardiogenic artifacts of the capnogram, and means for removing verified cardiogenic artifacts of the capnogram.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of embodiments systems and methods described below and are not meant to limit the scope of the invention in any manner, which scope shall be based on the claims appended hereto.

FIG. 1 illustrates an embodiment of a ventilator-capnograph system connected to a human patient.

FIG. 2 illustrates an embodiment of a method for monitoring the ventilation of a patient being ventilated by a medical ventilator-capnograph system.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. The reader will understand that the technology described in the context of a medical ventilator for human patients could be adapted for use with other systems such as ventilators for non-human patients and general gas transport systems.

Medical ventilators are used to provide a breathing gas to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gas having a desired concentration of oxygen is supplied to the patient at desired pressures and rates. Ventilators capable of operating independently of external sources of pressurized air are also available.

While operating a ventilator, it is desirable to control the percentage of oxygen in the gas supplied by the ventilator to the patient. Further, it is desirable to monitor the CO₂ levels in the respiration gas from the patient. Accordingly, ventilator systems may have capnographs for non-invasively determining the concentrations and/or pressures of CO₂ in the respiration gases of a patient, such as end tidal CO₂ or the amount of carbon dioxide released during exhalation and at the end of expiration (ETCO₂).

As known in the art, capnographs are devices for measuring and monitoring CO₂ in a gas stream. In one common design, the capnograph utilizes a beam of infra-red light, which is passed across the ventilator circuit and onto a sensor, to determine the level of CO₂ in a patient's respiration gasses. As the amount of CO₂ in the respiration gas increases, the amount of infra-red light that can pass through the respiration gas and onto the sensor decreases, which changes the voltage in a circuit. The sensor utilizes the change in voltage to calculate the amount of CO₂ contained in the gas. Other designs are known in the art and any capnography technology, now known or later developed, may be used in the embodiments described herein to obtain CO₂ readings.

Although ventilators and capnographs have been previously utilized on the same patient, ventilators typically display data based solely on ventilator data monitored by the ventilator. Further, capnographs typically display data based solely on the CO₂ readings. However, it is desirable to provide information that incorporates capnograph data with ventilator data to the patient, ventilator operator, and/or medical caregiver.

The present disclosure describes ventilator-capnograph systems and methods for monitoring the ventilation of a patient. The ventilator-capnograph systems described herein integrate capnographic data with ventilator data to provide the operator, medical caregiver, and/or the patient with more precise patient information for the treatment and ventilation of the patient.

An embodiment of the ventilator-capnograph systems described herein is a system that is capable of eliminating or substantially reducing the cardiogenic artifacts from a capnograph. As observed in several clinical cases, the action of the cardiac muscle or the pumping of the heart can cause enough volume change in the thorax to be interpreted as small flow changes by the capnometer sensor. These flow changes are induced by cardiogenic artifacts and may cause brief, periodic, low-amplitude disturbances of the capnogram (that is, the set of CO₂ data taken over time) and, if sufficiently large, can cause false ETCO₂ readings and also lead to an inappropriately high report of the volume of CO₂ per minute. The ventilator-capnograph system as described herein can be adapted to independently verify that these small flow changes or low-amplitude oscillatory disturbances in the capnogram are coincident with the pulse of a patient, allowing the operator and the ventilator to ignore these cardiogenic artifacts for the purposes of ETCO₂ detection and CO₂ volume per minute calculations.

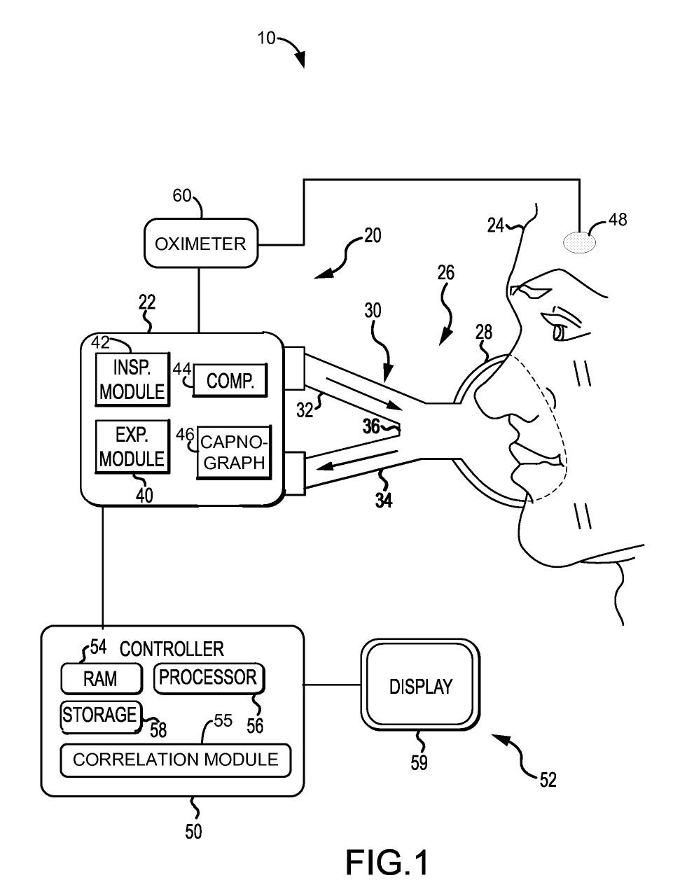

FIG. 1 illustrates an embodiment of a ventilator-capnograph system 10 attached to a human patient 24. The ventilator-capnograph system 10 includes a ventilator 20 in communication with a capnograph 46. As shown in FIG. 1 the capnograph 46 may be an integral part of ventilator 20. In an alternative embodiment, the capnograph 46 may be a separate component from ventilator 20.

Ventilator 20 includes a pneumatic gas delivery system 22 (also referred to as a pressure generating system 22) for circulating breathing gases to and from patient 24 via the ventilation tubing system 26, which couples the patient 24 to the pneumatic gas delivery system 22 via physical patient interface 28 and ventilator breathing circuit 30.

Ventilator breathing circuit 30 could be a dual-limb or single-limb circuit 30 for carrying gas to and from the patient 24. In a dual-limb embodiment as shown, a wye fitting 36 may be provided as shown to couple the patient interface 28 to the inspiratory limb 32 and the expiratory limb 34 of the ventilator breathing circuit 30. Examples of suitable patient interfaces 28 include a nasal mask, nasal/oral mask (which is shown in FIG. 1), nasal prong, full-face mask, tracheal tube, endotracheal tube, nasal pillow, etc.

Pneumatic gas delivery system 22 may be configured in a variety of ways. In the present example, system 22 includes an expiratory module 40 coupled with an expiratory limb 34 and an inspiratory module 42 coupled with an inspiratory limb 32. Compressor 44 or another source or sources of pressurized gas (e.g., pressured air and/or oxygen) is controlled through the use of one or more pneumatic gas delivery systems, such as a gas regulator.

Capnograph 46 is in data communication with ventilator 20. This communication allows the ventilator 20 and capnograph 46 to send data, instructions, and/or commands to each other. Capnograph 46 is in communication with processor 56 of ventilator 20.

Capnograph 46 monitors the concentrations of carbon dioxide in the respiratory gas with a carbon dioxide sensor located in the ventilator 20, such as in the breathing circuit 30, the patient connection port, or the capnograph 46 (e.g., the patient connection port or the expiratory side of the breathing system) via a side-stream capillary. The carbon dioxide sensor allows the capnograph 46 to monitor in real-time volumetric carbon dioxide (VCO₂), end-tidal carbon dioxide (ETCO₂), and minute volume. The capnograph 46 may generate a capnogram with these data. However, the action of the cardiac muscle or the pumping of the heart of patient 24 can cause enough volume change in the thorax of patient 24 to be interpreted as small flow changes by the carbon dioxide sensor. The reading of these flow changes is referred to herein as “cardiogenic artifacts”. If the movement of the thorax is large enough, it can lead to false ETCO₂ readings and also lead to an inappropriately high report of the volume of CO₂ per minute. Further, if the cardiogenic artifacts are large enough, they cause the capnogram generated by the capnograph 46 to exhibit brief, periodic, low-amplitude oscillatory disturbances. Typically, the larger the patient's 24 heart, the larger the cardiogenic artifacts.

However, these low-amplitude oscillatory disturbances must be within a predetermined threshold to have been caused by the volume changes caused by the pumping of the patient's cardiac muscle. For example, in one embodiment, if the cardiogenic artifacts or oscillations registering on the capnogram are above about 0.7 Hertz, the oscillations are likely from the volume changes caused by the pumping of the patient's cardiac muscle. Further, in this or another embodiment, if the oscillations registering on the capnogram have a frequency of less than 0.7 Hertz, the oscillations cannot be reasonably ascribed to the volume changes caused by the pumping of the patient's cardiac muscle alone.

Pneumatic gas delivery system 22 may include a variety of other components, including sources for pressurized air and/or oxygen, mixing modules, valves, sensors, tubing, filters, etc. In one embodiment, the pneumatic gas delivery system 22 includes a sensor 48. Sensor 48 is any sensor 48 suitable for monitoring the pulse rate or heart rate of patient 24. As used herein the terms “pulse rate” and “heart rate” are considered to be interchangeable in the present disclosure and in the claims. While “pulse rate” and “heart rate” refer to different measurements, it is understood by a person of skill in the art that either may be used for the purposes of this disclosure and for the purposes of the claims. In one embodiment, sensor 48 includes at least one of a cardiac monitor 48, an oximeter sensor 48, and/or a flow sensor 48 and pressure sensor 48. The readings from the flow sensor 48 and/or pressure sensor 48 may be utilized in combination with gender, weight, and height of patient 24 to monitor the pulse rate or heart rate of patient 24. In one embodiment, the operator inputs the gender, weight, and/or height of patient 24.

in one embodiment, as illustrated in FIG. 1, the ventilator-capnograph system 10 includes an oximeter 60. Oximeter 60 monitors the concentration of oxygen in the blood of patient 24 (e.g., as SpO₂) from data gathered with an oximeter sensor 48. The oximeter 60 is in communication with oximeter sensor 48.

As shown in FIG. 1, the oximeter 60 is a completely separate and independent component from ventilator 20. In an alternative embodiment, oximeter 60 is located inside of ventilator 20 and/or the pneumatic gas delivery system 22. As discussed above, the oximeter 60 and the ventilator 20 are in communication. This communication allows the ventilator 20 and the oximeter 60 to exchange data, commands, and/or instructions. In one embodiment, oximeter 60 is in communication with processor 56 of ventilator 20.

In one embodiment, the oximeter 60 monitors the pulse rate of patient 24 with oximeter sensor 48. The oximeter 60 monitors the pulse rate by monitoring the frequency of signal fluctuations caused by the expansion and contraction of the arterial blood vessels with each pulse as monitored by the oximeter sensor 48.

Controller 50 is in communication with pneumatic gas delivery system 22, capnograph 46, display 59, and an operator interface 52, which may be provided to enable an operator to interact with the ventilator 20 (e.g., change ventilator settings, select operational modes, view monitored parameters, etc.). Controller 50 may include memory 54, one or more processors 56, storage 58, and/or other components of the type commonly found in command and control computing devices.

The memory 54 is non-transitory computer-readable storage media that stores software that is executed by the processor 56 and which controls the operation of the ventilator 20. In an embodiment, the memory 54 comprises one or more solid-state storage devices such as flash memory chips. In an alternative embodiment, the memory 54 may be mass storage connected to the processor 56 through a mass storage controller (not shown) and a communications bus (not shown). Although the description of non-transitory computer-readable media contained herein refers to a solid-state storage, it should be appreciated by those skilled in the art that non-transitory computer-readable storage media can be any available media that can be accessed by the processor 56. Non-transitory computer-readable storage media includes volatile and non-volatile, removable and non-removable media implemented in any method or technology for storage of information such as non-transitory computer-readable instructions, data structures, program modules or other data. Non-transitory computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the processor 56.

In one embodiment, as illustrated in FIG. 1, the controller 50 further includes a correlation module 55. In alternative embodiment, not shown, the correlation module 55 is a separate component from or independent of controller 50. In another embodiment, not shown, the correlation module 55 is a separate component from or independent of ventilator 20.

The correlation module 55 identifies potential cardiogenic artifacts and correlates the potential cardiogenic artifacts with the pulse rate or heart rate of a patient 24. If correlation module 55 determines that the pulse rate or heart rate of patient 24 correlates with potential cardiogenic artifacts, the correlation module 55 removes or minimizes the distortions to the capnogram caused by the cardiogenic artifacts by adjusting the carbon dioxide data before it is used by the ventilator for display or when performing calculations. The correlation module 55 removes or minimizes the distortions to the capnogram caused by the cardiogenic artifacts by attempting to redraw or reconstruct the corrupted segment(s) of the capnogram as if there had been no cardiogenic disruption in the first place. If correlation module 55 determines that the pulse rate or heart rate of patient 24 does not correlate with potential cardiogenic artifacts, the correlation module 55 does not remove or adjust the data from carbon dioxide sensor readings.

Accordingly, the ventilator-capnograph system 10 as described herein verifies the likelihood that small CO₂ fluctuations or “distortions” of the capnogram are coincident with the pulse rate of patient 24, allowing the operator and/or the ventilator 20 to ignore these cardiogenic artifacts for the purposes of ETCO₂ detection and CO₂ volume per minute calculations. In one embodiment, the correlation module 55 performs the steps of identifying potential cardiogenic artifacts, correlating the potential cardiogenic artifacts, and verifying the cardiogenic artifacts simultaneously or at the same time. Accordingly, the correlation module 55 may perform these steps in real-time. In another embodiment, the correlation module 55 performs these steps in some other type of sequential order.

In one embodiment, correlation module 55 is activated upon user command. In an alternative embodiment, the correlation module 55 is activated based on a preset or a preselected ventilator setting. In another embodiment, correlation module 55 is activated repeatedly based on a preset or a preselected ventilator setting. In yet another embodiment, the correlation module 55 may always be active or may be activated based on data from the oximeter.

In the depicted example, operator interface 52 includes a display 59 that is touch-sensitive, enabling the display 59 to serve both as an input user interface and as an output device. In an alternative embodiment, the display 59 is not touch sensitive or an input user interface. The display 59 can display any type of ventilation information, such as sensor readings, parameters, commands, alarms, warnings, and/or smart prompts (i.e., ventilator-determined operator suggestions). Display 59 displays a capnogram to illustrate the concentrations of carbon dioxide in the respiratory gas of patient 24 being ventilated by ventilator 20. In one embodiment, display 59 displays a capnogram modified by correlation module 55 to exclude verified cardiogenic artifacts. In alternative embodiment, upon operator selection or command, display 59 displays a capnogram modified by correlation module 55 to exclude verified cardiogenic artifacts with the removed cardiogenic artifacts reinserted.

In an alternative embodiment, not shown, the capnograph 46 includes a display. In one embodiment, the capnograph display displays a capnogram modified by correlation module 55 to exclude verified cardiogenic artifacts. In an alternative embodiment, upon operator selection, the capnograph display displays the capnogram modified by correlation module 55 so that both the capnogram with and without the verified cardiogenic artifacts is shown.

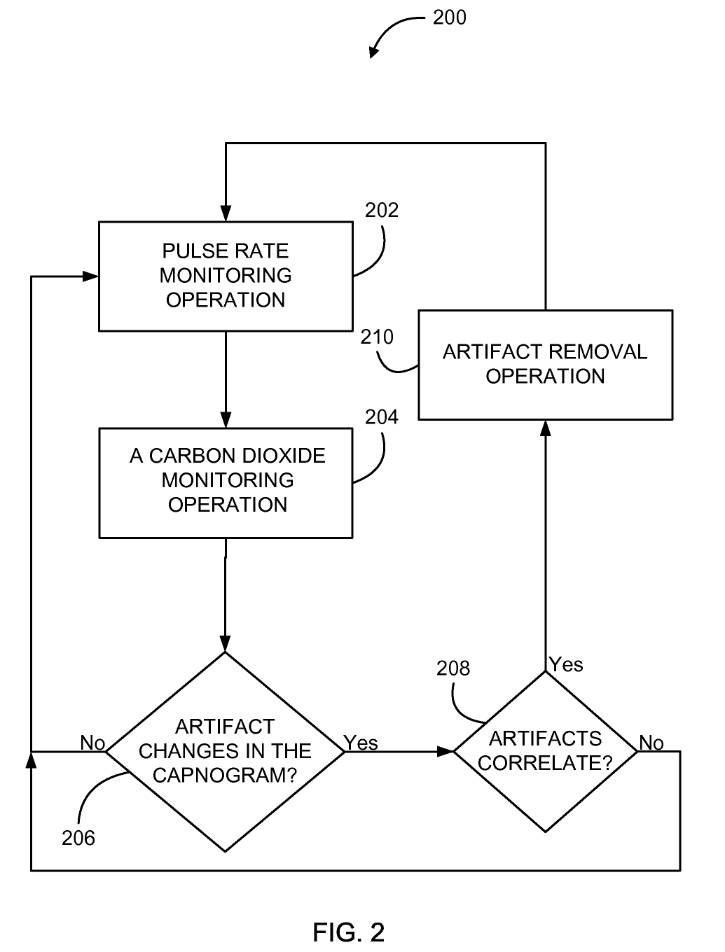

FIG. 2 illustrates an embodiment of a method 200 for monitoring a patient being ventilated by a medical ventilator-capnograph system. As illustrated, method 200 performs a pulse rate monitoring operation 202. Pulse rate monitoring operation 202 monitors a pulse rate or heart rate of a patient being ventilated by a medical ventilator-capnograph system with a sensor. The sensor is any sensor or combination of sensors suitable for monitoring the pulse rate or heart rate of the ventilator patient. In one embodiment, the sensors include a flow sensor and a pressure sensor. In an alternative embodiment the sensor is an oximeter sensor, pulse rate sensor or cardiac monitor. In any case, the pulse rate or heart rate of the ventilator patient is monitored using the sensor or sensors, possibly in combination with other data known to the ventilator such as the height, weight, and gender of the ventilator patient. In one embodiment, method 200 receives the height, weight, and gender of the patient from operator input.

In addition to monitoring the pulse rate, method 200 performs a carbon dioxide monitoring operation 204. Carbon dioxide monitoring operation 204 monitors the amount of carbon dioxide in the respiration gas of the ventilator patient with a capnograph. The capnograph utilizes a carbon dioxide sensor in the ventilator, such as in the breathing circuit, the patient connection port, or the capnograph (e.g., the patient connection port or the expiratory side of the breathing system) via a side-stream capillary to monitor the amount of carbon dioxide in the respiration gas from the ventilator patient. However, as noted above the action of the cardiac muscle or the pumping of the heart of the ventilator patient can cause enough volume change in the thorax of patient 24 to be interpreted as small flow changes by the carbon dioxide sensor. If the movement of the thorax is large enough, it can lead to false ETCO₂ readings and also lead to an inappropriately high report of the volume of CO₂ per minute. The capnograph utilizes the carbon dioxide sensor to monitor the carbon dioxide in the respiration gas of the ventilator patient to generate or derive a capnogram. If the cardiogenic artifacts are large enough, they appear in the capnogram as brief, periodic, low-amplitude disturbances that disrupt the expected capnogram trace.

It is understood by a person of skill in the art that the pulse rate monitoring operation 202 and the carbon dioxide monitoring operation 204 may be performed in any order and/or simultaneously. In one embodiment, the pulse rate monitoring operation 202 and/or the carbon dioxide monitoring operation 204 are performed in real-time.

Next, method 200 performs a first decision operation 206. First decision operation 206 determines if there are potential cardiogenic artifacts. The potential cardiogenic artifacts are any small, periodic, low-level oscillations that disrupt the capnogram. First decision operation 206 may be performed with a correlation module. If first decision operation 206 determines that the capnogram exhibits potential cardiogenic artifacts, first decision operation 206 selects to perform a second decision operation 208. If first decision operation 206 determines that the capnogram exhibits no potential cardiogenic artifacts, the method returns to the pulse rate monitoring operation 202.

In one embodiment, first decision operation 206 is performed upon user or operator command. In an alternative embodiment, first decision operation 206 is performed at a preset, preselected, and/or preconfigured time. In another embodiment, first decision operation 206 is performed continuously and/or repeatedly based on a preset, a preconfigured, and/or a preselected time duration.

If first decision operation 206 determines that the capnogram exhibits potential cardiogenic artifacts, method 200 performs a second decision operation 208. Second decision operation 208 may be performed with a correlation module. Second decision operation 208 determines if the capnogram exhibits potential cardiogenic artifacts that correlate with the monitored pulse rate of the ventilator patient. If second decision operation 208 determines that the potential cardiogenic artifacts correlate with the monitored pulse rate or heart rate of the patient, second decision operation 208 selects to perform artifact removal operation 210. Further, as soon as the potential cardiogenic artifacts are correlated with the monitored pulse rate or heart rate of the patient, the potential cardiogenic artifacts become verified cardiogenic artifacts. If second decision operation 208 determines that the potential cardiogenic artifacts do not correlate with the monitored pulse rate or heart rate of the patient, the method returns to the pulse rate monitoring operation 202.

In one embodiment, second decision operation 208 is performed upon user or operator command. In an alternative embodiment, second decision operation 208 is performed at a preset, preselected, and/or preconfigured time. In another embodiment, second decision operation 208 is performed continuously and/or repeatedly based on a preset, a preconfigured, and/or a preselected time duration.

It is understood by a person of skill in the art that the first decision operation 206 and the second decision operation 208 may be performed in any order and/or simultaneously. In one embodiment, the first decision operation 206 and/or the second decision operation 208 are performed in real-time.

The artifact removal operation 210 removes verified cardiogenic artifacts from the capnogram of the capnograph. The artifact removal operation 210 may be performed by the correlation module. In one embodiment, method 200 removes the verified cardiogenic artifacts from a carbon dioxide-related parameter derived from the monitoring of the carbon dioxide. In an embodiment, the carbon dioxide related parameter includes ETCO₂ and CO₂ volume per minute. Accordingly, method 200 as described herein verifies that small CO₂ fluctuations or oscillations of the capnogram are coincident with the pulse rate of a patient, allowing the operator and/or the ventilator to ignore these cardiogenic artifacts for the purposes of ETCO₂ detection and CO₂ volume per minute calculations.

In one embodiment, method 200 performs a display operation. Display operation displays a capnogram wherein the verified cardiogenic artifacts have been removed. The display operation may be performed by a ventilator display and/or by a capnograph display. In an additional embodiment, method 200 displays the capnogram with verified cardiogenic artifacts upon operator selection.

In one embodiment, method 200 is performed by the medical ventilator-capnograph system illustrated in FIG. 1 and described above.

In an alternative embodiment, a computer-readable medium having computer-executable instructions for performing methods for monitoring the ventilation of a patient being ventilated by a medical ventilator-capnograph system are disclosed. These methods include repeatedly performing the steps disclosed in method 200.

In another embodiment, a medical ventilator-capnograph system is disclosed. The medical ventilator-capnograph system includes: means for monitoring a pulse rate of a patient being ventilated by a medical ventilator-capnograph system; means for monitoring carbon dioxide in breathing gas from the patient to derive a capnogram; means for determining potential cardiogenic artifacts on a capnogram; means for correlating the potential cardiogenic artifacts of the capnogram with the pulse rate of the patient to verify cardiogenic artifacts of the capnogram; and means for removing verified cardiogenic artifacts of the capnogram. In another embodiment, the medical ventilator-capnograph system further includes means for displaying the capnogram without the verified cardiogenic artifacts. In an embodiment, the means for the medical ventilator-capnograph system are all illustrated in FIG. 1 and described in the above description of FIG. 1. However, the means described above for FIG. 1 and illustrated in FIG. 1 are exemplary only and are not meant to be limiting.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for calving out the described features and functions and interfaces, and those variations and modifications that may be made to the hardware or software or firmware components described herein as would be understood by those skilled in the art now and hereafter.

Numerous other changes may be made which will readily suggest themselves to those skilled in the art and which are encompassed in the spirit of the disclosure and as defined in the appended claims. While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for monitoring the ventilation of a patient being ventilated by a medical ventilator-capnograph system, the method comprising:
monitoring a pulse rate of a patient being ventilated by a medical ventilator-capnograph system with at least one of a flow sensor, a pressure sensor, a cardiac monitor, and an oximeter;
monitoring the patient with a capnograph, the capnograph monitors an amount of carbon dioxide in respiration gas from the patient to derive a capnogram;
determining potential cardiogenic artifacts of the capnogram;
correlating the potential cardiogenic artifacts of the capnogram with the pulse rate of the patient to verify cardiogenic artifacts of the capnogram; and
removing verified cardiogenic artifacts of the capnogram.

2. The method of claim 1, further comprising displaying a capnogram wherein the verified cardiogenic artifacts have been removed.

3. The method of claim 2, wherein the capnogram is displayed by at least one of a capnograph display and a ventilator display.

4. The method of claim 1, further comprising receiving height, weight, and gender of the patient from operator input.

5. The method of claim 4, further comprising:
monitoring the pulse rate of the patient with the flow sensor and the pressure sensor based on the height, weight, and gender of the patient.

6. The method of claim 1, further comprising:
monitoring the pulse rate of the patient with the oximeter.

7. The method of claim 1, further comprising:
monitoring the pulse rate of the patient with the cardiac monitor.

8. The method of claim 1, further comprising:
displaying the capnogram with verified cardiogenic artifacts reinserted upon operator selection.

9. The method of claim 1, further comprising:
removing the verified cardiogenic artifacts from a carbon dioxide related parameter derived from the amount of carbon dioxide in the respiration gas from the patient.

10. The method of claim 9, wherein the carbon dioxide related parameter is ETCO₂ and CO₂ volume per minute.

11. The method of claim 1, further comprising:
correcting a monitored volumetric carbon dioxide (VCO₂) for errors based on the verified cardiogenic artifacts.

12. A medical ventilator-capnograph system, comprising:
a pneumatic gas delivery system, the pneumatic gas delivery system adapted to control a flow of gas from a gas supply to a patient via a ventilator breathing circuit;
at least one sensor, the at least one sensor monitors a pulse rate of the patient;
a capnograph, the capnograph monitors an amount of carbon dioxide in respiration gas from the patient in the ventilator breathing circuit to generate a capnogram;
a correlation module, the correlation module is adapted to identify potential cardiogenic artifacts of the capnogram, correlate the potential cardiogenic artifacts with the pulse rate of the patient, and remove verified cardiogenic artifacts of the capnogram; and
a processor in communication with the pneumatic gas delivery system, at least one sensor, the capnograph, and the correlation module.

13. The medical ventilator-capnograph system of claim 12, further comprising a display in communication with processor, the display is adapted to display the capnogram.

14. The medical ventilator-capnograph system of claim 12, further comprising an oximeter in communication with the processor, the oximeter monitors the pulse rate of the patient.

15. The medical ventilator-capnograph system of claim 12, wherein the at least one sensor is a flow sensor and a pressure sensor.

16. The medical ventilator-capnograph system of claim 12, wherein the at least one sensor is a cardiac monitor.

17. The medical ventilator-capnograph system of claim 12, wherein the at least one sensor is an oximeter sensor.

18. A computer-readable medium having computer-executable instructions for performing a method for monitoring the ventilation of a patient being ventilated by a medical ventilator-capnograph system, the method comprising:
repeatedly monitoring a pulse rate of a patient being ventilated by a medical ventilator-capnograph system;
repeatedly monitoring carbon dioxide in breathing gas from the patient to derive a capnogram;
repeatedly determining potential cardiogenic artifacts of a capnogram;
repeatedly correlating the potential cardiogenic artifacts of the capnogram with the pulse rate of the patient to verify cardiogenic artifacts of the capnogram; and
repeatedly removing verified cardiogenic artifacts of the capnogram.

19. The computer-readable medium of claims 18, further comprising:
repeatedly displaying the capnogram without the cardiogenic artifacts.

20. A medical ventilator-capnograph system, comprising:
means for monitoring a pulse rate of a patient being ventilated by a medical ventilator-capnograph system;
means for monitoring carbon dioxide in breathing gas from the patient to derive a capnogram;
means for determining potential cardiogenic artifacts of a capnogram;
means for correlating the potential cardiogenic artifacts of the capnogram with the pulse rate of the patient to verify cardiogenic artifacts of the capnogram; and
means for removing verified cardiogenic artifacts of the capnogram.

21. The medical ventilator-capnograph system of claim 20, further comprising:
means for displaying the capnogram without the cardiogenic artifacts.
